(19) United States
(12) Patent Application Publication
(10) Pub. No.: US 2016/0002275 A1
(43) Pub. Date: Jan. 7, 2016

(54) PROCESS FOR PREPARATION AND PURIFICATION OF CANAGLIFLOZIN

(71) Applicant: CADILA HEALTHCARE LIMITED, Ahmedabad (IN)

(72) Inventors: Sanjay Jagdish DESAI, Ahmedabad (IN); Jayprakash Ajitsingh PARIHAR, Ahmedabad (IN); Jagdish Maganlal PATEL, Ahmedabad (IN); Uday Suresh SURYAWANSHI, Ahmedabad (IN); Jaisukh Bhupatbhai BHALALA, Ahmedabad (IN)

(73) Assignee: CADILA HEALTHCARE LIMITED, Ahmedabad (IN)

(21) Appl. No.: 14/755,263

(22) Filed: Jun. 30, 2015

(30) Foreign Application Priority Data
Jul. 3, 2014 (IN) 2153/MUM/2014
Aug. 5, 2014 (IN) 2514/MUM/2014

(51) Int. Cl. C07H 1/06; C07H 7/04

(52) U.S. Cl. CPC C07H 1/06; C07H 7/04

(57) ABSTRACT

The present invention provides a canagliflozin complex. The complex is provided with an adsorbent selected from activated carbon, silica gel, ionic or non-ionic polymer and a cyclodextrin or derivatives thereof. The invention also provides a process for the preparation of canagliflozin and its intermediates thereof. The invention further provides a process for the purification of canagliflozin.

### FIELD OF THE INVENTION

The field of the invention relates to a process for the preparation of (2S,3R,4R,5S,6R)-2-(3-((5-(4-fluorophenyl)thiophen-2-yl)methyl)-4-methylphenyl)-6-(hydroxyethyl)tetrahydro-2H-pyran-3,4,5-triol. In particular, the present invention relates to a process for the preparation of canagliflozin and its intermediates thereof. More particularly, the present invention relates to a process for the purification of canagliflozin.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

(2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol is also known as Canagliflozin, is an inhibitor of subtype 2 sodium-glucose transport protein (SGLT2) which is chemically represented as compound of Formula (I).

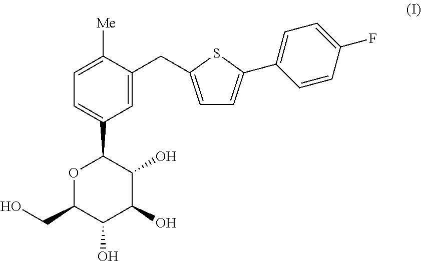 [SMILES: Cc1ccc([C@@H]2O[C@H](CO)[C@@H](O)[C@H](O)[C@H]2O)cc1Cc1ccc(-c2ccc(F)cc2)s1]

U.S. Pat. No. 7,943,788 B2 discloses canagliflozin and a process for its preparation.

U.S. Pat. No. 7,943,582 B2 (the '582 patent) discloses crystalline form of 1-(β-D-glucopyranosyl)-4-methyl-3-[5-(4-fluorophenyl)-2-thienylmethyl]benzene hemihydrate and process for preparation thereof.

U.S. PG-Pub. No. 2011/0212905 discloses crystalline form of 1-(β-D-glucopyranosyl)-4-methyl-3-[5-(4-fluorophenyl)-2-thienylmethyl]benzene hemihydrate and process for preparation thereof.

U.S. PG-Pub. Nos. 2009/0233874, 2010/099883 and 2008/0146515 discloses similar process for the preparation of canagliflozin substantially as same as shown in scheme-1 below.

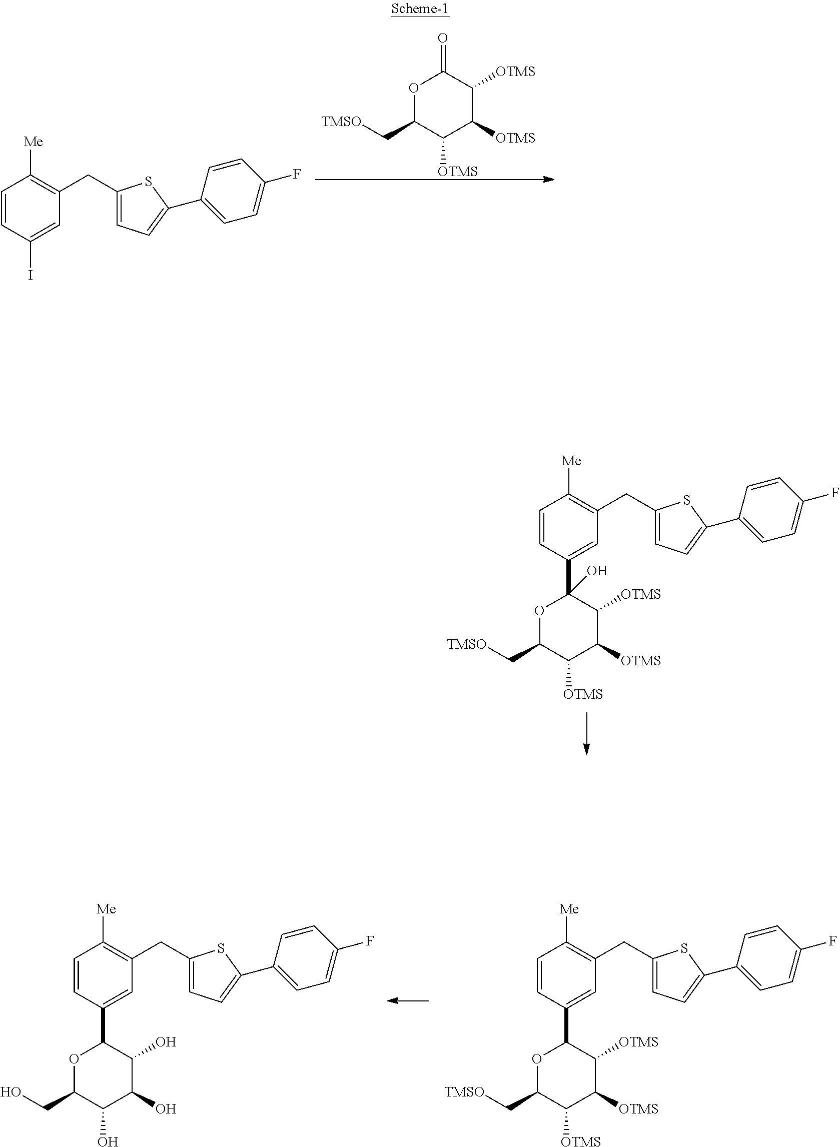

International (PCT) Publication No. WO 2011/079772 discloses a process for the preparation of canagliflozin by reduction of keto group of acetyl protected compound followed by hydrolysis.

U.S. PG-Publication No. 2014/0128595 discloses a process for the preparation of canagliflozin from anhydroglucopyranose derivative substantially as same as shown in scheme-2 below.

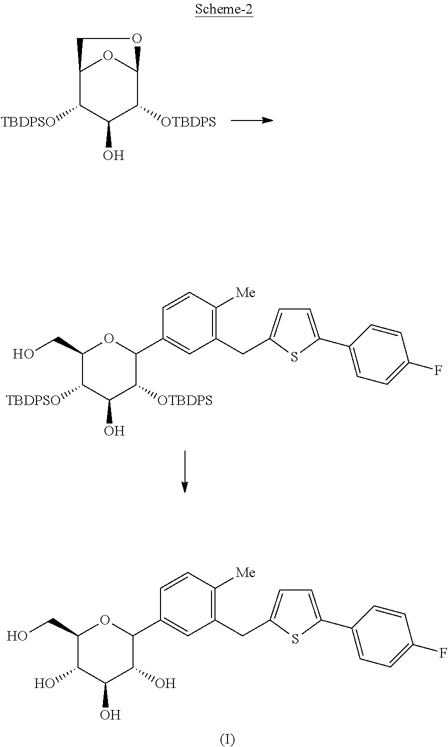

The prior-art processes requires sequence of protection/deprotection of canagliflozin obtained in the course of the reactions and further purification or crystallization to obtain canagliflozin in reasonably pure form. This sequences of processes results in high amount of yield loss.

In view of the above prior art, there is provided a novel, efficient and convenient process for preparation of canagliflozin which is at least a useful alternative to the prior art as well as an efficient and convenient method for purification of canagliflozin without sequence of protection and deprotection.

### SUMMARY OF THE INVENTION

In one general aspect, there is provided a canagliflozin complex with an adsorbent selected from activated carbon, silica gel, ionic or non-ionic polymer and a cyclodextrin or derivatives thereof useful for the purification of canagliflozin.

In another general aspect, there is provided a process for the preparation of canagliflozin, the process comprising:
(a) reacting a compound of Formula (V) with a compound of Formula (IV),

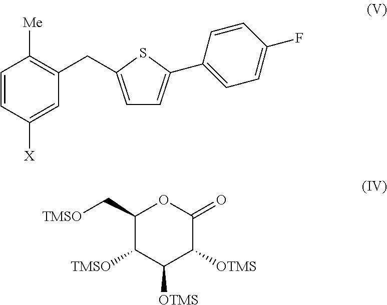

wherein X═Br or I,
followed by in-situ deprotection to obtain a compound of Formula (III),

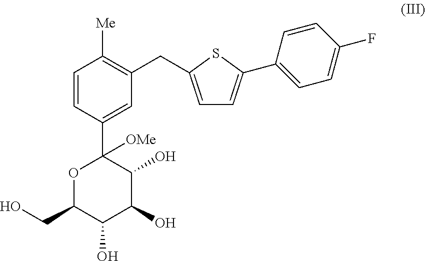 [SMILES: COC1(c2ccc(C)c(Cc3ccc(-c4ccc(F)cc4)s3)c2)O[C@H](CO)[C@@H](O)[C@H](O)[C@H]1O]

(b) reacting the compound of Formula (III) with an appropriate molar equivalent of a silane reagent to obtain a compound of Formula (II);

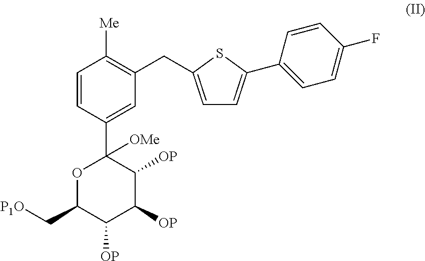

wherein P═H or P₁; and P₁=hydroxy protecting group,
(c) reducing the compound of Formula (II) with a reducing agent to obtain the canagliflozin; and
(d) optionally, purifying the canagliflozin by forming a complex with an adsorbent.

In another general aspect, there is provided a compound of Formula (IIa).

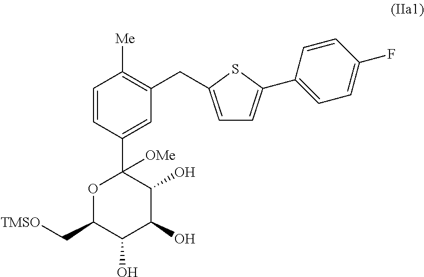

In another general aspect, there is provided a process for purification of canagliflozin, the process comprising:
(a) providing a solution of canagliflozin in one or more solvents to obtain the solution,
(b) contacting the solution with an adsorbent to obtain canagliflozin-adsorbent complex;
(c) treating the canagliflozin-adsorbent complex with one or more solvents; and
(d) obtaining the pure canagliflozin by the removal of the solvent.

In another general aspect, there is provided a pharmaceutical composition comprising canagliflozin prepared by the process of the present invention and one or more of pharmaceutically acceptable excipients, diluents and carriers.

### DETAILED DESCRIPTION OF THE INVENTION

The above and other objects of the present invention are achieved by the process of the present invention, which leads to canagliflozin suitable for pharmaceutical use.

As used herein, the terms such as “reacting”, “protecting”, “reducing” have meanings as widely used by general prior art in the field of invention and can be easily understood by those skilled in the art.

As used herein, the term “deprotection” or “deprotecting” used herein means removing the protecting group(s) to obtain a compound with free hydroxy.

As used herein, the term “pure canagliflozin” used herein means canagliflozin obtained after the purification by forming complex with an adsorbent having purity of about 96% or more, of about 97% or more, of about 98% of more, more particularly of about 99% or more and most particularly of about 99.5% of more.

As used herein, the term “crystalline eanagliflozin-β-cyclodextrin complex” means the canaglifiozin-β-cyclodextrin complex having of about 60% or more of percentage crystailinity, of about 70% or more, of about 80% or more, of about 90% or more, when measured by x-ray powder diffraction pattern.

In general, the reactions of present inventions are performed under inert atmosphere wherever required due to the nature of the reaetant(s), reagent(s) or catalyst(s) used therein.

As used herein, the terms “obtaining” means isolating the canagliflozin by way of filtration, filtration under vacuum, centrifugation, decantation and the like. The product obtained may be further or additionally dried to achieve the desired moisture values. For example, the product may be dried in a tray drier, dried under vacuum and/or in a Fluid Bed Drier.

In one general aspect, there is provided a canagliflozin complex with an adsorbent selected from activated carbon, silica gel, ionic or non-ionic polymer and a cyclodextrin or derivatives thereof.

In another general aspect, there is provided a crystalline canagliflozin complex with an adsorbent selected from one or more of cyclodextrin.

In another general aspect, there is provided a crystalline canagliflozin-β-cyclodextrin complex.

In another general aspect, there is provided a process for the preparation of canagliflozin, the process comprising:
(a) reacting a compound of Formula (V) with a compound of Formula (IV),

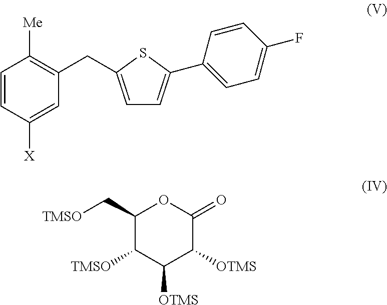

wherein X═Br or I,
followed by in-situ deprotection to obtain a compound of Formula (III),

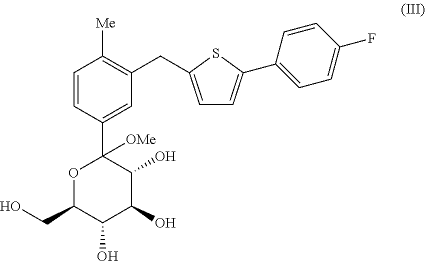 [SMILES: COC1(c2ccc(C)c(Cc3ccc(-c4ccc(F)cc4)s3)c2)O[C@H](CO)[C@@H](O)[C@H](O)[C@H]1O]

(b) reacting the compound of Formula (III) with an appropriate molar equivalent of a silane reagent to obtain a compound of Formula (II); and

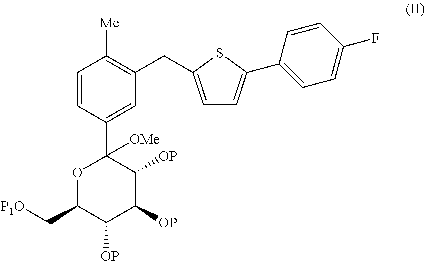

wherein P═H or P₁; and P₁=hydroxy protecting group,
(c) reducing the compound of Formula (II) with a reducing agent to obtain the canagliflozin; and
(d) purifying the canagliflozin by forming a complex with an adsorbent.

In general, the reaction further comprises
(a) reacting the compound of Formula (III) with about 1-2 molar equivalent of a silane reagent to obtain a compound (IIa1); or

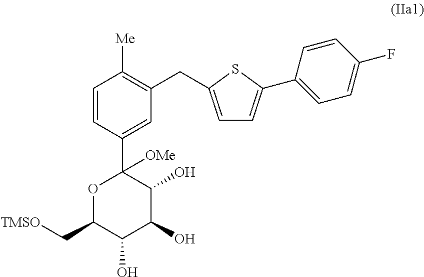

(b) reacting the compound of Formula (III) with about 2-6 molar equivalent of a silane reagent to obtain a compound (IIb1)

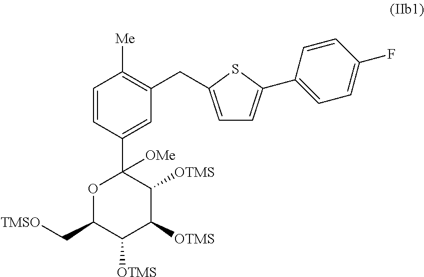

wherein the silane reagent is selected from one or more of trimethylsilane, triethylsilane, t-butyl-dimethylsilane, t-butyl-diphenylsilane and triphenylsilane.

In another general aspect, there is provided a process for the preparation of canagliflozin, the process comprising:
(a) reacting a compound of Formula (V) with a compound of Formula (IV)

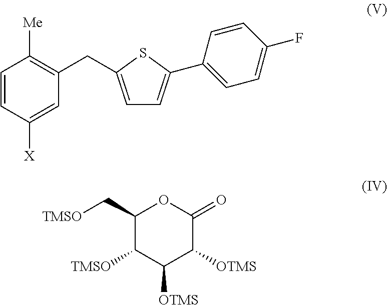

wherein X═Br or I,
in the presence of an alkyl lithium or alkyl magnesium halide reagent to obtain a compound of Formula (IIIa),

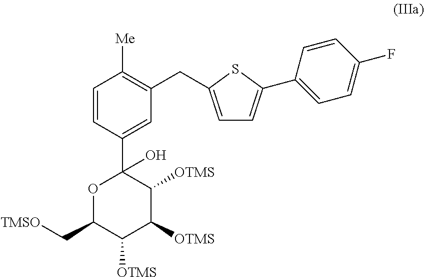

(b) in-situ reacting the compound (IIIa) with methane sulfonic acid to obtain a compound of Formula (III),

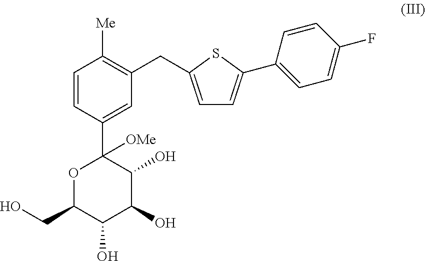 [SMILES: COC1(c2ccc(C)c(Cc3ccc(-c4ccc(F)cc4)s3)c2)O[C@H](CO)[C@@H](O)[C@H](O)[C@H]1O]

(c) reacting the compound of Formula (III) with about 1-2 molar equivalent of a silane reagent selected from one or more of trimethylsilane, triethylsilane, t-butyl-dimethylsilane, t-butyl-diphenylsilane and triphenylsilane to obtain a compound of Formula (IIa);

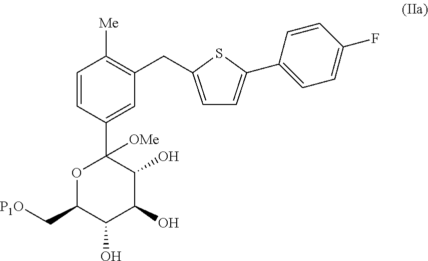

wherein P1 is hydroxy protecting group.
(d) reducing the compound of Formula (IIa) with a reducing agent to obtain the canagliflozin; and
(e) purifying the canagliflozin by forming a complex with an adsorbent to obtain the pure canagliflozin.

In another general aspect, there is provided a process for the preparation of canagliflozin, the process comprising:
(a) reacting a compound of Formula (V) with a compound of Formula (IV)

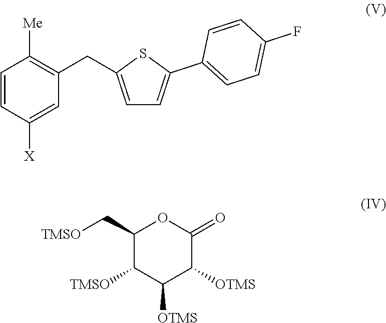

wherein X═Br or I,
in the presence of an alkyl lithium or alkyl magnesium halide reagent to obtain a compound of Formula (IIIa),

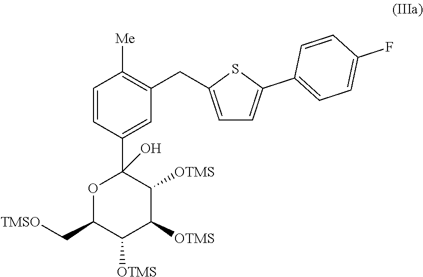

(b) in-situ reacting the compound (IIIa) with methane sulfonic acid to obtain a compound of Formula (III),

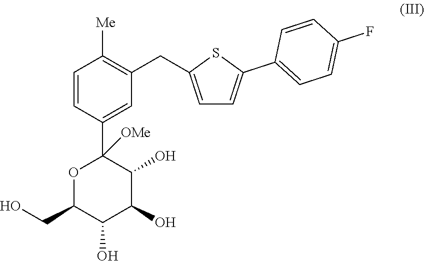 [SMILES: COC1(c2ccc(C)c(Cc3ccc(-c4ccc(F)cc4)s3)c2)O[C@H](CO)[C@@H](O)[C@H](O)[C@H]1O]

(c) reacting the compound of Formula (III) with about 2-6 molar equivalent of a silane reagent selected from one or more of trimethylsilane, triethylsilane, t-butyl-dimethylsilane, t-butyl-diphenylsilane and triphenylsilane to obtain a compound of Formula (IIb);

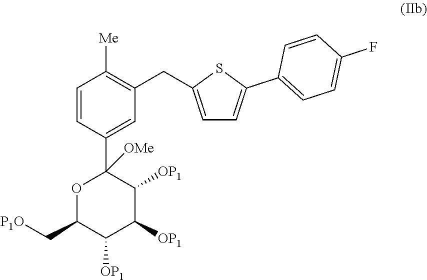

wherein P1 is hydroxy protecting group.
(d) reducing the compound of Formula (IIb) with a reducing agent to obtain the canagliflozin; and
(e) purifying the canagliflozin by forming a complex with an adsorbent to obtain the pure canagliflozin.

In another general aspect, there is provided a compound of Formula (IIa1).

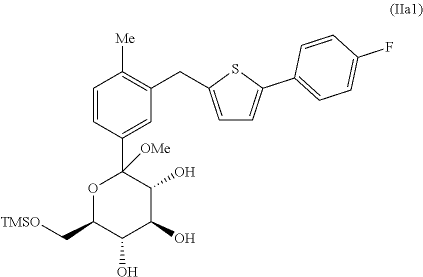

In another general aspect, there is provided a use of compound of Formula (IIa1) for the preparation of canagliflozin.

In general, the hydroxy protecting group P₁ of all above embodiments may be same or different and individually selected from a group comprising methoxymethyl (MOM), -t-butyloxy, -t-butyldimethylsilyl (TBDMS), -t-butyl diphenylsilyl (TBDPS), trimethylsilyl (TMS), acetate, pivalate, and benzoate.

In general, the reaction of the compound of Formula V and the compound of Formula IV to obtain compound of Formula III is carried out in the presence of an alkyl lithium reagent comprises of one or more of methyl lithium, ethyl lithium, n-butyl lithium, Sec-butyl lithium, t-butyl lithium and (trimethylsilyl)-methyl lithium and an alkyl magnesium halide comprises of one or more of ethyl magnesium bromide, isopropyl magnesium chloride and methyl magnesium iodide.

In general, an additional lithium salt is optionally added during the reaction to obtain compound of Formula (III) with better stereoselectivity.

In general, the compound of Formula (III) is prepared by reacting the compound of Formula (IV) and the compound of Formula (V) in the presence of n-butyl lithium to obtain compound of Formula (IIIa) followed by in-situ reaction with methane sulfonic acid.

The reaction of compound (IV) and compound (V) is carried out in one or more of inert solvent selected from diethyl ether, diisopropyl ether, methyl tert-butyl ether, tetrahydrofuran, 1,4-dioxane, toluene, xylene, n-hexane, n-heptane, cyclohexane or mixture thereof. In particular, mixture of tetrahydrofuran and toluene is used.

In general, the reaction of compound (IIIa) with methane sulfonic acid is carried out in one or more of alcohols selected from methanol, ethanol, isopropanol, n-butanol and t-butanol; esters selected from methyl acetate, ethyl acetate, isopropyl acetate and butyl acetate; ketones selected from acetone, methyl ethyl ketone and methyl isobutyl ketone. In particular, methanol is used.

In general, the compound of Formula (III) may be converted to compound of Formula IIa or IIb using sufficient molar equivalence of the reagent used for protecting the hydroxy group.

In general, the compound of Formula (III) may be converted to compound of Formula (IIa) using about 1-2 molar equivalent of a silane at reagent selected from one or more of trimethylsilane, triethylsilane, t-butyl-dimethylsilane, t-butyl-diphenylsilane and triphenylsilane. In general, the silane reagent may be used at 0.9-1.9 molar equivalents, particularly 1.0-1.7 molar equivalents, more particularly 1.1-1.5 molar equivalents or most particularly 1.2-1.3 molar equivalents.

In general, the compound of Formula (III) may be converted to compound of Formula (IIb) by using 2-6 molar equivalent of a silane as reagent selected from trimethylsilane, triethylsilane, t-butyl-dimethylsilane, t-butyl-diphenylsilane and triphenylsilane. In general, the silane reagent may be used at 3-6 molar equivalents, particularly 4-6 molar equivalents, more particularly 5.1-5.9 molar equivalents or most particularly 5.2-5.5 molar equivalents.

In general, in addition to the silane reagent, a catalyst may also be added to provide highly favorable conditions for this reaction. In particular, trimethylsilane is used with N-methylmorpholine (NMM) or 4-(Dimethylamino)pyridine.

In general, the reducing agent comprises one or more of Zn(Hg)/HCl, NH₂NH₂/KOH, LiAlH₄, NaBH₄, Pd(C), Ni and trialkylsilanes (triethylsilane, triphenylsilane).

In particular, triethylsilane may be used with titanium tetrachloride, aluminium tetrachloride, boron trifluoride diethyl etherate or trifluoroacetic acid in dichloromethane or tetrahydrofuran to obtain canagliflozin from compounds of Formula II, (IIa) or (IIb).

In general, the organic solvents used for the process of the present invention comprises one or more of alcohols selected from methanol, ethanol, isopropanol, 2-propanol, 1-butanol, and t-butyl alcohol; ketones selected from acetone, butanone and methyl isobutyl ketone; esters selected from ethyl acetate, isopropyl acetate, t-butyl acetate and isobutyl acetate, chlorinated hydrocarbons selected from methylene dichloride, ethylene dichloride and chlorobenzene, acetonitrile, and polar aprotic solvents selected from N,N-dimethylformamide, N,N-dimethyl acetamide, N-methylpyrrolidone and dimethylsulfoxide.

In another general aspect, there is provided use of a canagliflozin-β-cyclodextrin complex for the preparation of amorphous canagliflozin.

In another general aspect, there is provided a process for purification of canagliflozin, the process comprising:
(a) providing a solution of canagliflozin in one or more solvents to obtain the solution,
(b) contacting the solution with an adsorbent to obtain canagliflozin-adsorbent complex;
(c) treating the canagliflozin-adsorbent complex with one or more solvents; and
(d) obtaining the pure canagliflozin by the removal of the solvent.

In general, the solution of canagliflozin may be obtained in the course of its synthesis which may be a reaction mass containing canagliflozin after determination of completion of a reaction or a solution containing canagliflozin obtained after primary isolation and/or work-up of the reaction mass obtained. The canagliflozin compound is obtained by removing solvent from such a solution or dissolving the residue obtained after removal of the solvent again in the same or different solvent to use for further steps.

In general, any solid state form of canagliflozin may be used to prepare the solution. The solid state form may be any crystalline or amorphous form or any co-crystal or co-precipitate form of canagliflozin.

The process parameters include adding one or more adsorbent to the solution of canagliflozin, obtained by any of the method described herein above, in one or more solvents and obtaining pure canagliflozin by the removal of the solvent.

In general, the adsorbent comprises one or more of activated carbon, silica gel, ionic or non-ionic polymer, a cyclodextrin or derivatives thereof.

The activate carbon and silica gel comprises various grades thereof of varying pore and particle size and their nature viz. acidic, basic or neutral.

The ionic or non-ionic polymer comprises one or more of carboxymethyl cellulose, hydroxypropylmethyl cellulose, hydroxypropylmethyl cellulose acetate succinate, methacrylic acid copolymers, polyvinylalcohol and polyvinylpyrrolidones (PVP) selected from K-15, K-30, K-60, K-90 and K-120.

The cyclodextrin comprises one or more of α-cyclodextrin, β-cyclodextrin and γ-cyclodextrin; methyl β-cyclodextrin, 2-hydroxypropylated β- and γ-cyclodextrins, sulfobutylated-β-cyclodextrin, branched cyclodextrins (glucosyl- and maltosyl-β-cyclodextrins), acetylated β- and γ-cyclodextrins and sulfated cyclodextrins.

In general, the solvent comprises one or more of methanol, ethanol, n-propanol, i-propanol, n-butanol, i-butanol, t-butanol, acetone, methylethylketone, methylisobutylketone, diethylether, diisopropylether, methyl t-butyl ether, tetrahydrofuran, dioxane, 1,2-dimethoxyethane, methylformate, ethylacetate, isopropylacetate, butylacetate, acetonitrile, dimethylformamide, dimethylacetamide, N-methylpyrrolidone, dimethylsulfoxide, methylene dichloride, ethylene dichloride, chloroform, carbon tetrachloride, pentane, hexane, n-heptane, octane, cyclohexane, benzene, toluene, mix-xylene, m-xylene, o-xylene, p-xylene and petroleum ether or mixture thereof.

The pure canagliflozin may be obtained from the solution containing canagliflozin and the adsorbent by the removal of the solvent. The solvent may be removed by one or more methods comprising filtration, decantation, evaporation, distillation, freeze drying, agitated thin film drying and spray drying.

In general, when the adsorbent is insoluble in the solvent, pure canagliflozin is obtained by removing the solvent from the solution by filtration or decantation; and when the adsorbent is soluble in the solvent, than pure canagliflozin is obtained by partial or complete removal of solvent by distillation, evaporation, freeze drying, agitated thin film drying or spray drying and adding solvent in which the adsorbent is insoluble and obtaining the pure canagliflozin by filtration or decantation.

In general, the canagliflozin compound obtained as per the processes discloses in the prior-art provides the canagliflozin with purity of about 80-90% as measured by HPLC and is obtained in the form of an oily mass or a foamy material, which is further subjected to acetylation protection and deprotection followed by recrystallization to obtain pure canagliflozin. The process of the present invention provides a canagliflozin compound having purity of more than 99% from the compound having purity of about 80-90%. as measured by HPLC without such tedious processes.

It has been found that activated carbon, silica gel and cyclodextrin complex are very much effective in removing certain polar as well non-polar impurities which are difficult to remove by simple crystallization purification processes. Varying grades of activated carbon may be used to eliminate certain specific polar or non-polar impurities.

Similar results as obtained from activated carbon are expected from varying the grades of silica, polymers and cyclodextrins.

The results of purity by HPLC of canagliflozin obtained are summarized in following Table.

| Input | Adsorbent | After isolation |
|---|---|---|
| 80-90% | β-cyclodextrin | 96-98% |
| 80-90% | Hydroxypropyl | 96-98% |
| | methyl cellulose | |
| 80-90% | Activated carbon | 96-98% |
| 80-90% | β-cyclodextrin + | 97-99% |
| | Activated carbon | |
| 80-90% | β-cyclodextrin + | 97-99% |
| | Silica | |

In general, the present invention may be illustrated substantially as same as shown in Scheme-3.

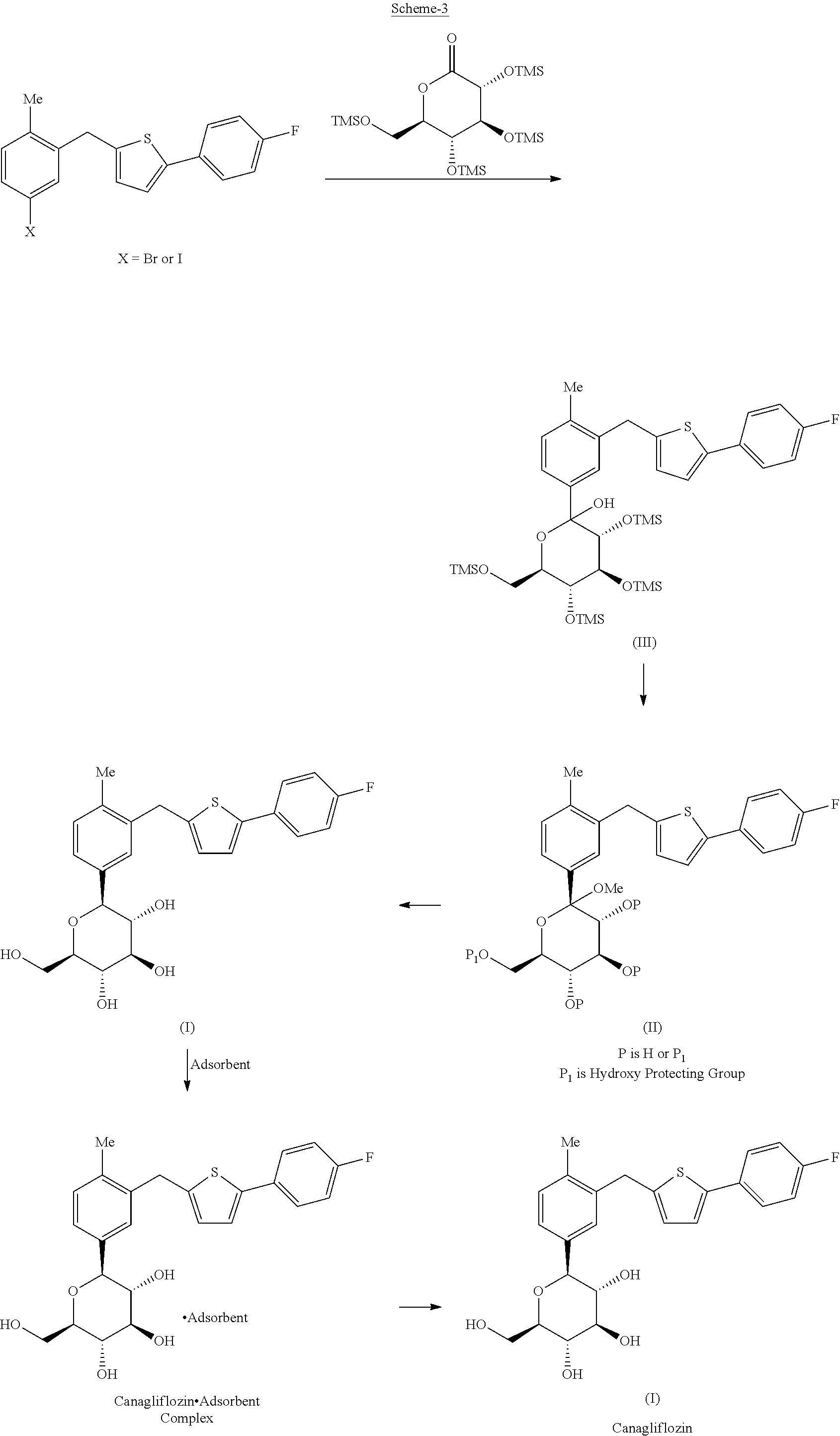

In another general aspect, there is provided a pharmaceutical composition comprising an amorphous canagliflozin prepared by the process of the present invention having one or more of pharmaceutically acceptable excipients, diluents or carriers.

The invention also encompasses pharmaceutical compositions comprising amorphous canagliflozin of the present invention. As used herein, the term “pharmaceutical compositions” or “pharmaceutical Formulations” includes tablets, pills, powders, liquids, suspensions, emulsions, granules, capsules, suppositories, or injection preparations.

Pharmaceutical compositions containing the canagliflozin of the present invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

The present invention is further illustrated by following examples which is provided merely to exemplify the invention and do not limit the scope of it.

### EXAMPLES

### Example-1

### Preparation of (3R,4S,5S,6R)-2-(3-((5-(4-fluorophenyl)thiophen-2-yl)methyl)-4-methylphenyl)-6-(hydroxymethyl)-2-methoxytetrahydro-2H-pyran-3,4,5-triol (III)

In 500 mL three necked round bottomed flask equipped with mechanical stirrer, thermometer and addition funnel were added 2-(5-bromo-2-methylbenzyl)-5-(4-fluorophenyl)thiophene (Va) (5 g) and 150 mL toluene at 25° C. 1.5 mL (1.6M) n-butyl lithium in hexane was added dropwise at room temperature and the solution was stirred for 30 minutes. This solution was cooled to −78° C. and added dropwise to a solution of 3,4,5-tris((trimethylsilyl)oxy)-6-(((trimethylsilyl)oxy)methyl)tetrahydro-2H-pyran-2-one (IV) (6.4 g) in 100 mL toluene and the mixture was stirred for 3 hours. The reaction mixture was treated with 2.5 g methanesulfonic acid in 100 mL methanol and stirred for 1 hour. The reaction mass was warmed to 25° C. and then added to pre-cool saturated sodium bicarbonate solution and resulting mass was extracted with ethyl acetate. The extract was washed with brine, dried over Na₂SO₄ and evaporated under reduced pressure to obtain compound of Formula (III).

### Example-1A

### Preparation of (3R,4S,5S,6R)-2-(3-((5-(4-fluorophenyl)thiophen-2-yl)methyl)-4-methylphenyl)-6-(hydroxymethyl)-2-methoxytetrahydro-2H-pyran-3,4,5-triol (III)

In 500 mL three necked round bottomed flask equipped with mechanical stirrer, thermometer and addition funnel were added 2-(5-bromo-2-methylbenzyl)-5-(4-fluorophenyl)thiophene (Va) (5 g) and 150 mL toluene at 25° C. 1.5 mL (1.6M) n-butyl lithium in hexane was added dropwise at room temperature and the solution was stirred for 30 minutes. This solution was cooled to −78° C. and added dropwise to a solution of 3,4,5-tris((trimethylsilyl)oxy)-6-(((trimethylsilyl)oxy)methyl)tetrahydro-2H-pyran-2-one (IV) (6.4 g) in 100 mL toluene and the mixture was stirred for 3 hours. The reaction mixture was treated with 2.5 g methanesulfonic acid in 100 mL methanol and stirred for 1 hour. The reaction mixture warmed to room temperature and stirred for 8 hours. Saturated sodium bicarbonate solution was added to the reaction mixture and the separated aqueous layer was extracted with toluene. The organic layer was distilled to remove toluene and the residue was dissolved in 50 mL methylene dichloride, washed with brine, dried over Na₂SO₄ and evaporated under reduced pressure to obtain residue. The residue was treated with 150 mL diisopropyl ether and stirred at 55° C. for 30 min, cooled, filtered and washed with diisopropyl ether to obtain compound of Formula (III).

### Example-1B

### Preparation of (3R,4S,5S,6R)-2-(3-((5-(4-fluorophenyl)thiophen-2-yl)methyl)-4-methylphenyl)-6-(hydroxymethyl)-2-methoxytetrahydro-2H-pyran-3,4,5-triol (III)

In 5 L three necked round bottomed flask equipped with mechanical stirrer, thermometer and addition funnel were added 100 g 2-(5-iodo-2-methylbenzyl)-5-(4-fluorophenyl)thiophene (Vb), 114.35 g 3,4,5-tris((trimethylsilyl)oxy)-6-(((tri-methylsilyl)oxy)methyl)tetrahydro-2H-pyran-2-one (IV), 2 L toluene and 1 L tetrahydrofuran at 30° C. The reaction mixture was cooled to −78° C. and 171.45 mL n-butyl lithium in hexane (1.6M) was added and the solution was stirred for 3 hours. The reaction mixture was treated with 94.16 g methanesulfonic acid in 1500 mL methanol and stirred for 1 hour. The reaction mixture warmed to 25° C. and stirred for 8 hours. The reaction mixture was cooled to 5° C. and saturated sodium bicarbonate solution was added to the reaction mixture and stirred for 30 min. The separated aqueous layer was extracted with toluene. The organic layer was distilled to remove toluene and the residue was dissolved in 300 mL methylene dichloride and 200 g silica gel of 60-120 mesh was added. The reaction mixture was stirred for 30 min at 30° C., washed with brine, dried over Na₂SO₄ and evaporated under reduced pressure to obtain residue. The residue was treated with 1 L diisopropyl ether and stirred at 55° C. for 30 min, cooled, filtered and washed with diisopropyl ether to obtain compound of Formula (III).

### Example-2A

### Preparation of (3R,4S,5S,6R)-2-(3-((5-(4-fluorophenyl)thiophen-2-yl)methyl)-4-methylphenyl)-2-methoxy-6-(((trimethylsilyl)oxy)methyl)tetrahydro-2H-pyran-3,4,5-triol (IIa1)

In 500 mL three necked round bottomed flask equipped with mechanical stirrer, thermometer and addition funnel were added 10 g compound of Formula (III), 80 mL methylene dichloride and 4.3 g N-methylmorpholine at −5 to 5° C. 2.7 g trimethylsilyl chloride was added slowly and stirred for 1 hour. After confirming the reaction completion TLC, 30 mL pre-cool water was slowly added, stirred and layers were separated. The separated aqueous layer was extracted with methylene dichloride and the combined organic layers were washed with 20% sodium dihydrogen phosphate dihydrate solution, water and brine. The organic layer was evaporated under reduced pressure to obtain compound of Formula (IIa).

### Example-2B

### Preparation of (3R,4S,5S,6R)-2-(3-((5-(4-fluorophenyl)thiophen-2-yl)methyl)-4-methylphenyl)-2-methoxy-6-(((trimethylsilyl)oxy)methyl)tetrahydro-2H-pyran-3,4,5-triol (IIa1)

In 1 L three necked round bottomed flask equipped with mechanical stirrer, thermometer and addition funnel were added 100 g compound of Formula (III) and 900 mL methanol at 30° C. and stirred for 1 hour. The reaction mixture was filtered to remove silica gel and washed with methanol. The filtrate was distilled under vacuum to remove methanol completely, 350 mL methylene dichloride and 42.63 g N-methylmorpholine were added to the residue and cooled to at −5 to 5° C., 34.34 g trimethylsilyl chloride was lot-wise added and stirred for 45 min. After confirming the reaction completion TLC, 300 mL pre-cool water was slowly added, stirred and layers were separated. The separated aqueous layer was extracted with methylene dichloride and the combined organic layers were washed with 20% sodium dihydrogen phosphate dihydrate solution, water and brine. The separated organic layer was dried over sodium sulfate and filtered to obtain compound of Formula (IIa1).

### Example-3A

### Preparation of Canagliflozin of Formula (I)

In 1 L three necked round bottomed flask equipped with mechanical stirrer, thermometer and addition funnel was added solution of compound (IIa) prepared in example-2B and cooled to −70° C. 8 mL triethylsilane and 5.5 mL boron trifluoridediethyl etherate were added dropwise within 1 hour maintaining the reaction temperature between −70° C. The reaction was warmed to −30° C. and stirred for 30 min. The reaction mixture was then added to freshly prepared sodium bicarbonate solution at 5° C. and then allowed to warm to room temperature and stirred for 20 mints to adjust the pH of 7-8. The reaction mass was then slowly added to cold water. The resulting mass was extracted with ethyl acetate. The combined organic layers were washed with saturated bicarbonate solution, dried over Na₂SO₄ and evaporated under reduced pressure to obtain canagliflozin having purity 86% by HPLC.

### Example-3B

### Preparation of Canagliflozin of Formula (I)

In 2 L three necked round bottomed flask equipped with mechanical stirrer, thermometer and addition funnel was added the solution of compound (IIa) prepared in example-2B and cooled to −70° C. 67.38 g triethylsilane and 83.08 g boron trifluoridediethyl etherate were added dropwise within 1 hour maintaining the reaction temperature between −70° C. The reaction was warmed to −30° C. and stirred for 3 hours. The reaction mixture was then added to freshly prepared sodium bicarbonate solution at 5° C. and then allowed to warm to room temperature and stirred for 20 mints to adjust the pH of 7-8. The reaction mixture was then slowly added to cold water. The separated aqueous layer was extracted with 200 mL methylene dichloride. The combined organic layer was washed with 300 mL water and distilled completely to remove methylene dichloride. The resulting residue extracted with 500 mL ethyl acetate and stirred to obtain clear solution. The reaction mixture was treated with brine and saturated bicarbonate solution to separate the layers. The separated organic layer was dried over sodium sulfate, charcoalized and filtered. The filtrate is distilled to remove ethyl acetate completely under vacuum. The residue was dissolved in 300 mL methylene dichloride and 200 g silica gel of 60-120 mesh was added. The reaction mixture was stirred for 30 min at 30° C. and distilled completely under reduced pressure to obtain residue. The residue was treated with 500 L diisopropyl ether and stirred at 55° C. for 30 min, cooled, filtered and washed with diisopropyl ether to obtain canagliflozin (I) having purity 87% by HPLC.

### Example-4

### Preparation of (3R,4S,5R,6R)-2-(3-((5-(4-fluorophenyl)thiophen-2-yl)methyl)-4-methylphenyl)-2-methoxy-6-(((trimethylsilyl)oxy)methyl)tetrahydro-2H-pyran-3,4,5-triyl)tris(oxy)tris(trimethylsilane) (IIb1)

In 500 mL three necked round bottomed flask equipped with mechanical stirrer, thermometer and addition funnel were added 10 g compound of Formula (III), 100 mL methylene dichloride and 15 g N-methylmorpholine at 0 to 5° C. 12.7 g trimethylsilyl chloride was added slowly and stirred for 1 hour. After confirming the reaction completion by TLC, 300 mL pre-cool water was slowly added, stirred and layers were separated. The separated aqueous layer was extracted with methylene dichloride and the combined organic layers were washed with 20% sodium dihydrogen phosphate dihydrate solution, water and brine. The organic layer was evaporated under reduced pressure to obtain compound of Formula (IIb1).

### Example-5

### Preparation of Canagliflozin

In 500 mL three necked round bottomed flask equipped with mechanical stirrer, thermometer and addition funnel was added 20 g compound (IIb1) prepared in example-4 and 100 mL methylene dichloride at −25° C. to −30° C. 11 mL triethylsilane and 7.8 mL boron trifluoridediethyl etherate was added drop wise within 1-2 hours maintaining the reaction temperature between −25° C. to −30° C. The reaction was stirred for 30 min and then allowed to warn to room temperature and stirred for 1.5-2 hours. The reaction mixture was then slowly added to cold water. The reaction mixture was extracted with ethyl acetate. The combined organic layers were washed with saturated bicarbonate solution, dried over sodium sulfate and evaporated under reduced pressure to obtain canagliflozin having purity 86% by HPLC.

### Example-6

### Purification of Canagliflozin

In 250 L three necked round bottomed flask equipped with mechanical stirrer, thermometer and addition funnel 10 g canagliflozin (purity 85%) and 100 mL toluene were stirred to obtain a clear solution. 10 g Polyvinylpyrrolidone was added to the solution and stirred for 2-3 hours. The reaction mixture was filtered and washed with toluene. The solid was stirred in ethyl acetate and water mixture for 30 min. The separated ethyl acetate layer was evaporated to dryness to obtain pure canagliflozin. (7.1 g. Purity 96.55% by HPLC).

### Example-7

### Purification of Canagliflozin

In 250 L three necked round bottomed flask equipped with mechanical stirrer, thermometer and addition funnel 10 g canagliflozin (purity 87%) and 100 mL toluene were stirred in in a round bottom flask to obtain a clear solution. 10 g β-cyclodextrin was added to the solution and stirred for 2-3 hours. The reaction mixture was filtered and washed with toluene. The solid was stirred in ethyl acetate and water mixture for 30 min. The separated ethyl acetate layer was treated with activated carbon, filtered and evaporated to dryness to obtain pure canagliflozin. (7.9 g, Purity 98.93% by HPLC).

### Example-8

### Purification of Canagliflozin

In 250 L three necked round bottomed flask equipped with mechanical stirrer, thermometer and addition funnel 10 g canagliflozin (purity 87%) and 0.25 g activated carbon were stirred in 100 mL toluene for 15-20 min and filtered. 10 g β-cyclodextrin was added to the filtrate and stirred for 2-3 hours. The reaction mixture was filtered and washed with toluene. The solid was stirred in isopropyl acetate and water mixture for 30 min. The separated isopropyl acetate layer was evaporated to dryness to obtain pure canagliflozin. (7.7 g, Purity 99.12% by HPLC).

### Example-9

### Purification of Canagliflozin

In 250 L three necked round bottomed flask equipped with mechanical stirrer, thermometer and addition funnel, 10 g canagliflozin (purity 87%) and 100 mL toluene were stirred to obtain a clear solution. 10 g hydroxy propyl methyl cellulose was added to the solution and stirred for 2-3 hour. The reaction mixture was filtered, washed with toluene. The solid was stirred in isopropyl acetate and water mixture for 30 min and dried to obtain pure canagliflozin. (Purity 97-98% by HPLC).

### Example-10

### Purification of Canagliflozin

In 2 L three necked round bottomed flask equipped with mechanical stirrer, thermometer and addition funnel, 100 g canagliflozin (purity 87%) obtained in example-3B and 900 mL methanol were stirred for 45 min at 30° C. The reaction mixture was filtered to remove silica gel. The filtrate was distilled under vacuum completely below 45° C. 400 mL toluene was added and heated to 55° C. to obtain a clear solution. The reaction mixture was filtered and the filtrate was added 100 g β-cyclodextrine. The reaction mixture was heated at 75° C. for 30 min and cooled to 30° C. and further stirred for 30 min. 5 g canagliflozin β-cyclodextrin complex was added to the solution and further cooled to 5° C. The reaction mixture was stirred for 3 hours and filtered. The wet-cake was treated with 300 mL isopropyl acetate and heated at 75° C. for 30 min. The reaction mixture was cooled to 30° C. and stirred for 6 hours and further cooled to 5° C. and stirred for 3 hours. The reaction mixture was filtered and washed with isopropyl acetate and dried at 30° C. to obtain crystalline canagliflozin β-cyclodextrine complex having 40 g pure canagliflozin with 99% purity by HPLC.

### Example-11

### Preparation of Amorphous Canagliflozin

In 1 L three necked round bottomed flask equipped with mechanical stirrer, thermometer and addition funnel, 100 g canagliflozin β-cyclodextrine (purity 98%) obtained in example-10 and 400 mL acetone were stirred for 30 min at 30° C. The reaction mixture was filtered to remove β-cyclodextrine. The filtrate was distilled under vacuum completely below 45° C. 400 mL acetone was added to the residue to get clear solution at 30° C. 5 g activated charcoal was added and stirred for 20 min. The reaction mixture was filtered and the filtrate was spray dried using JISL Mini spray drier LSD-48 keeping feed pump at 30 rpm, inlet temperature at 60° C., outlet temperature at 40° C. and 2 Kg/cm2 hot air supply. The product was collected from cyclone and is further dried at 40° C.±5° C. under vacuum for 12 hours to get 80 g of amorphous canagliflozin having 99.6% purity by HPLC.

While the present invention has been described in terms of its specific embodiments, certain modifications and equivalents will be apparent to those skilled in the art and are intended to be included within the scope of the present invention.

1. A canagliflozin complex with an adsorbent selected from activated carbon, silica gel, ionic or non-ionic polymer and a cyclodextrin or derivatives thereof.

2. The canagliflozin complex according to claim 1 is crystalline wherein the adsorbent is selected from one or more of cyclodextrin.

3. A crystalline canagliflozin-β-cyclodextrin complex.

4. A process for the preparation of canagliflozin, the process comprising:
(a) reacting a compound of Formula (V) with a compound of Formula (IV), 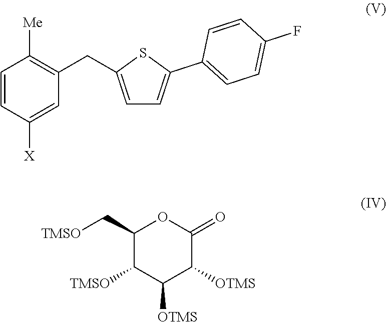
wherein X═Br or I,
followed by in-situ deprotection to obtain a compound of Formula (III). 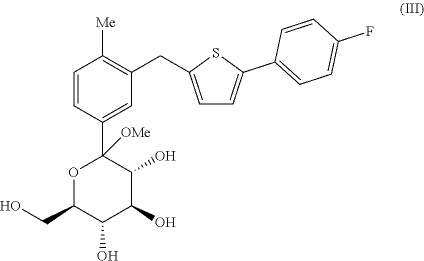 [SMILES: COC1(c2ccc(C)c(Cc3ccc(-c4ccc(F)cc4)s3)c2)O[C@H](CO)[C@@H](O)[C@H](O)[C@H]1O]
(b) reacting the compound of Formula (III) with an appropriate molar equivalent of a silane reagent to obtain a compound of Formula (II); 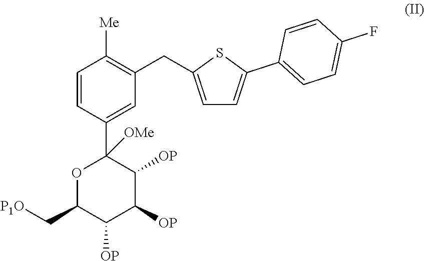
wherein P═H or P₁; and P₁=hydroxy protecting group,
(c) reducing the compound of Formula (II) with a reducing agent to obtain the canagliflozin; and
(d) optionally, purifying the canagliflozin by forming a complex with an adsorbent.

5. The process according to claim 4, wherein the process further comprises
(a) reacting the compound of Formula (III) with about 1-2 molar equivalent of a silane reagent to obtain a compound (IIa); or 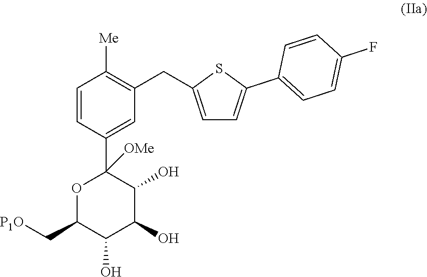
wherein P1 is hydroxy protecting group,
(b) reacting the compound of formula (III) with about 2-6 molar equivalent of a silane reagent to obtain a compound (IIb) 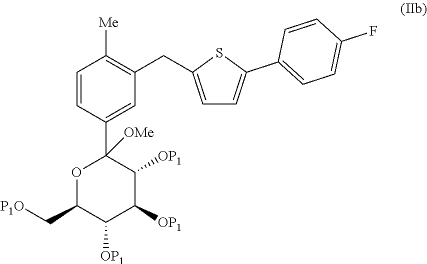
wherein P1 is hydroxy protecting group,
wherein the silane reagent is selected from one or more of trimethylsilane, triethylsilane, t-butyl-dimethylsilane, t-butyl-diphenylsilane and triphenylsilane.

6. The process for the preparation of pure canagliflozin, the process comprising:
(a) reacting a compound of Formula (V) with a compound of Formula (IV) 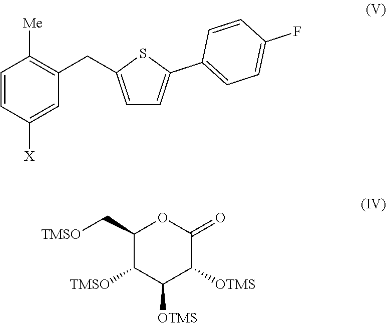
wherein X═Br or I.
in the presence of an alkyl lithium or alkyl magnesium halide reagent to obtain a compound of Formula (IIIa), 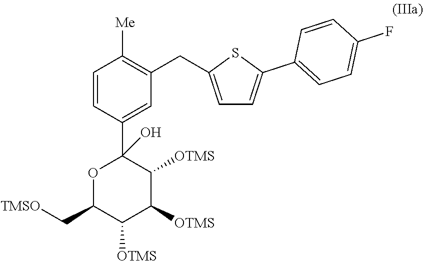
(b) in-situ reacting the compound (IIIa) with methane sulfonic acid to obtain a compound of Formula (III), 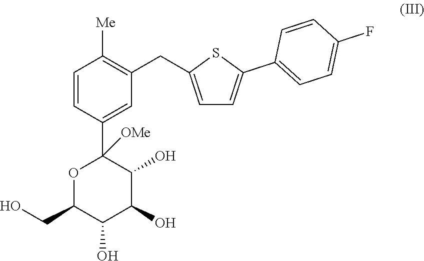 [SMILES: COC1(c2ccc(C)c(Cc3ccc(-c4ccc(F)cc4)s3)c2)O[C@H](CO)[C@@H](O)[C@H](O)[C@H]1O]
(c) reacting the compound of Formula (III) with about 1-2 molar equivalent of a silane reagent selected from one or more of trimethylsilane, triethylsilane, t-butyl-dimethylsilane, t-butyl-diphenylsilane and triphenylsilane to obtain a compound of Formula (IIa); 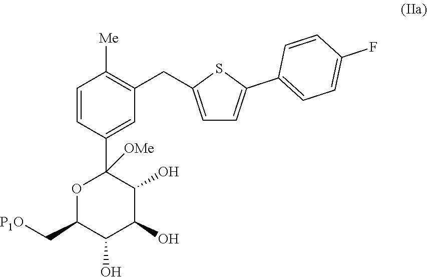
wherein P1 is hydroxy protecting group
(d) reducing the compound of Formula (IIa) with a reducing agent to obtain the canagliflozin; and
(e) purifying the canagliflozin by forming a complex with an adsorbent to obtain the pure canagliflozin.

7. A compound of Formula (IIa1).

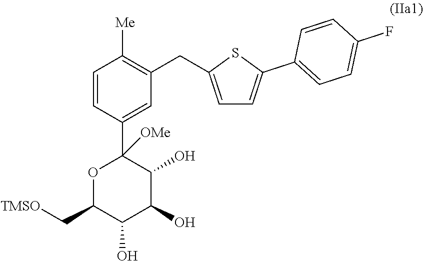

8. The process according to claim 6, wherein the alkyl lithium reagent comprises one or more of methyl lithium, ethyl lithium, n-butyl lithium, Sec-butyl lithium, t-butyl lithium and (trimethylsilyl)methyl lithium and an alkyl magnesium halide comprises of on or more of ethyl magnesium bromide, isopropyl magnesium chloride and methyl magnesium iodide.

9. The process according to claim 4, wherein the reaction in step (a) is carried out in one or more inert solvent selected from diethyl ether, diisopropyl ether, methyl, tert-butyl ether, tetrahydrofuran, 1,4-dioxane, toluene, xylene, n-hexane, n-heptane, cyclohexane or mixture thereof.

10. The process according to claim 6, wherein the reaction with methane sulfonic acid in step (b) is carried out in one or more of alcohols selected from methanol, ethanol, isopropanol, n-butanol and t-butanol; esters selected from methyl acetate, ethyl acetate, isopropyl acetate and butyl acetate; ketones selected from acetone, methyl ethyl ketone and methyl isobutyl ketone.

11. The process according to claim 6, wherein the 1-2 molar equivalent of silane reagent in step (c) is from about 1.2-1.3.

12. The process according to claim 4, wherein the reducing agent comprises one or more of Zn(Hg)/HCl, NH₂NH₂/KOH, LiAlH₄, NaBH₄, Pd(C), Ni and trialkylsilanes (triethylsilane, triphenylsilane).

13. The process according to claim 6, wherein the purification of canagliflozin in step (e) further comprises:
(a) providing a solution of canagliflozin in one or more solvents to obtain the solution,
(b) contacting the solution with an adsorbent to obtain canagliflozin-adsorbent complex;
(c) treating the canagliflozin-adsorbent complex with one or more solvents; and
(d) obtaining the pure canagliflozin by the removal of the solvent.

14. The process according to claim 13, wherein the solvent comprises one or more of methanol, ethanol, n-propanol, i-propanol, n-butanol, i-butanol, t-butanol, acetone, methylethylketone, methylisobutylketone, diethylether, diisopropylether, methyl t-butyl ether, tetrahydrofuran, dioxane, 1,2-dimethoxyethane, methylformate, ethylacetate, isopropylacetate, butylacetate, acetonitrile, dimethylformamide, dimethylacetamide, N-methylpyrrolidone, dimethylsulfoxide, methylene dichloride, ethylene dichloride, chloroform, carbon tetrachloride, pentane, hexane, n-heptane, octane, cyclohexane, benzene, toluene, mix-xylene, m-xylene, o-xylene, p-xylene and petroleum ether or mixture thereof.

15. The process according to claim 13, wherein the adsorbent comprises one or more of activated carbon, silica gel, ionic or non-ionic polymer, a cyclodextrin or derivatives thereof.

16. The process according to claim 13, wherein the solvent is removed by one or more methods comprising filtration, decantation, evaporation, distillation, freeze drying, agitated thin film drying and spray drying.

17. The crystalline canagliflozin-β-cyclodextrin complex according to claim 3 used for the preparation of amorphous canagliflozin.

18. A pharmaceutical composition of amorphous canagliflozin according to claim 17 having one or more pharmaceutically acceptable excipients, diluents or carriers.

19. The process according to claim 6, wherein the reaction in step (a) is carried out in one or more inert solvent selected from diethyl ether, diisopropyl ether, methyl tert-butyl ether, tetrahydrofuran, 1,4-dioxane, toluene, xylene, n-hexane, n-heptane, cyclohexane or mixture thereof.

20. The process according to claim 6, wherein the reducing agent comprises one or more of Zn(Hg)/HCl, NH₃NH₂/KOH, LiAlH₄, NaBH₄, Pd(C), Ni and trialkylsilanes (triethylsilane, triphenylsilane).
